(19) United States
(12) Patent Application Publication
(10) Pub. No.: US 2021/0355480 A1
(43) Pub. Date: Nov. 18, 2021

(54) Method For Creating Reference Cell Lines With Simultaneous Genetic Variants And Accurate Quantification Of Alelle Frequency

(71) Applicants: Yabin Lu, Pleasanton, CA (US); Gang Li, Albany, CA (US)

(72) Inventors: Yabin Lu, Pleasanton, CA (US); Gang Li, Albany, CA (US)

(21) Appl. No.: 17/214,839

(22) Filed: Mar. 27, 2021

Related U.S. Application Data
(60) Provisional application No. 63/005,484, filed on Apr. 6, 2020.

(51) Int. Cl. C12N 15/10; C12N 9/22; C12N 15/11; C12N 15/90; C12N 15/64; C12Q 1/6844

(52) U.S. Cl. CPC C12N 15/10; C12N 9/22; C12N 15/11; C12Q 1/6806; C12N 15/64; C12Q 1/6844; C12N 2310/20; C12N 15/902

(57) ABSTRACT

A method to simultaneously create multiple SNV, INDEL or fusion sequences harbored in a single cell line is provided. The method uses the CRISPR/Cas9 gene editing system to generate large sequence knock-in cell lines in an AAVS1 locus, or other safe harbor sites. Also provided is a method that allows specifically engineered quantitative marker sequences to accurately reflect copy numbers of inserted SNV, INDEL and fusion sequences. These methods allow accurate measurement of ratio or allele frequencies of genetic variants in a cell.

### CROSS-REFERENCE TO RELATED APPLICATIONS

This application claims priority to and the benefit of the provisional patent application titled “Method For Creating Reference Cell Lines With Simultaneous Genetic Variants And Accurate Quantification Of Allele Frequency”, application No. 63/005,484, filed in the United States Patent and Trademark Office on Apr. 6, 2020. The specification of the above referenced patent application is incorporated herein by reference in its entirety.

### SEQUENCE LISTING

The instant application contains a Sequence Listing which has been submitted electronically in ASCII format and is hereby incorporated by reference in its entirety. The ASCII copy, created on Jun. 10, 2021, is named Anchormolecular_Sequence_Listing_Jun_10_2021_Final.txt and is 4000 bytes in size.

### FIELD OF THE INVENTION

The present invention provides a method to simultaneously create multiple Single Nucleotide Variant (SNV), insertions or deletions (INDEL), or fusion sequences harbored in a single cell line. The method uses the Clustered Regularly Interspaced Short Palindromic Repeats (CRISPR)/Cas9 gene editing system to generate large sequence knock-in cell lines in an Adeno-associated virus integration site 1 (AAVS1) locus, or other safe harbor sites. The present invention also provides a method that allows specifically engineered quantitative marker sequences to accurately reflect copy numbers of inserted SNV, INDEL and fusion sequences.

### BACKGROUND OF THE INVENTION

Cell lines harboring clinically relevant genetic variants or mutations are critical reference material for oncology biopsy and genetic-based diagnostics. However, technologies utilizing Clustered Regularly Interspaced Short Palindromic Repeats (CRISPR) to create Single Nucleotide Variant (SNV), insertions or deletions (INDEL), fusions or structural variant sequences have been expensive and slow in meeting demand Hence there is a long felt but unresolved need for a method to simultaneously create multiple SNV, INDEL, fusions or structural variant sequences harbored in a single cell line.

For making reference standards and quality control samples for monitoring the performance of cell free deoxyribonucleic acid (cfDNA) or circulating tumor deoxyribonucleic acid (ctDNA) assays, gene fragments with the mutations are mixed with their wild-type gene counterparts at certain predetermined allele frequencies (AF %), which are expressed as a percentage of number of copies of mutant (variant of a gene) fragments over total fragments of the same allele. The mutant and the wild-type gene sequence can be distinguished by polymerase chain reaction (PCR) based detection methods exploiting the melting temperature difference resulting from the mutation. However, because of the high degree of sequence similarity between a wild-type DNA and the mutant bearing either a single nucleotide polymorphism (SNP) or a short insertion or deletion of a few nucleotides (small INDEL), the mutant DNA fragments are difficult to distinguish from their wild-type DNA, especially when the AF % is near or below about 1-2%. This makes it difficult to accurately make the cfDNA reference standard or quality controls at the low AF %, which is often critical for detection of ctDNAs often in low copies in a patient sample. Hence, there is a long felt but unresolved need for a method that allows specifically engineered quantitative marker sequences to accurately reflect copy numbers of the inserted SNV, INDEL and fusions or structural variant sequences.

### BRIEF DESCRIPTION OF THE DRAWINGS

FIG. 1 illustrates the cloning synthesized gene fragment sequence into the vector.

### DETAILED DESCRIPTION OF THE INVENTION

Disclosed herein is a method to simultaneously create multiple SNV, INDEL, fusions or structural variant sequences harbored in a single cell line. One example of the method uses a CRISPR/CRISPR-associated protein 9 (Cas9) gene editing system to generate large sequence knock-in cell lines in an Adeno-Associated Virus Integration Site 1 (AAVS1) locus, or other safe harbor sites. The chromosomal “safe harbor sites” (SHS) are intragenic or extragenic regions of human genome that are able to accommodate predictable expression of newly integrated DNA without disrupting the expression of adjacent or more distant genes which adversely affect a host cell or organism. These putative SHS help in developing effective gene therapies; in the investigation of gene structure, function, and regulation; and in cell-based biotechnology. The safe harbor sites comprise, but are not limited to AAVS1, human gene trap (ROSA)26S or locus (hROSA26), and C—C chemokine receptor type 5 (CCR5).

The procedure for generating large sequence knock-in cell lines in an Adeno-Associated Virus Integration Site 1 (AAVS1) locus, or other safe harbor sites is as follows:

Step 1: Constructing donor plasmid and the guide ribonucleic acid RNA (gRNA)

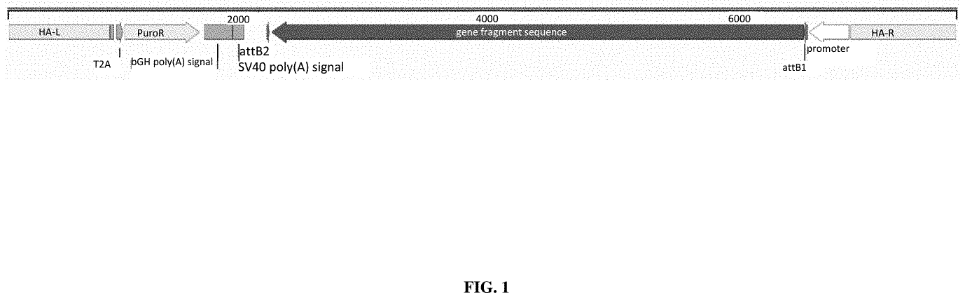

This step comprises:
a) Determining a safe harbor gene to insert in the gene fragment sequence.
b) Designing and cloning the gRNA targeting the safe harbor gene.
c) Synthesizing a large piece of gene fragment sequence containing target sequences.
d) Constructing a vector containing both sides of homologous recombination sequences at a CRISPR/Cas9 cutting site in the safe harbor gene, puromycin resistance gene, and promoters.
e) Cloning synthesized gene fragment sequence into the vector as shown in FIG. 1.

Step 2: Performing CRISPR/Cas 9 gene knock-in of cytosine-adenine-guanine (CAG) gene fragment sequence to a specific cell line.

This step comprises:
a) Selecting the gRNA with the best cutting efficiency for the knock-in, in which both gRNA plasmid and donor vector are transfected into cells by either chemical transfection or electroporation.
b) Selecting the transfected cells for specific antibiotic resistant (here puromycin resistant) cells that receive the donor plasmid in a target region. After selection, the transfected cells are expanded for checking the presence of correct gene insertion.

Step 3: Generating and screening single cell clone

This step takes 1-2 months and comprises:
a) Sorting the cloned gene fragment sequence into 96-well plates. A series of dilutions are performed to achieve single clone/per well. To screen for 100 single cell clones, at least three 96-well plates are required. Each well is observed daily for the first 3 days to record whether a single cell is seeded. About ten to fifteen days are needed for the cell expansion.
b) Extracting genomic deoxyribonucleic acid (DNA) from each cell clone for polymerase chain reaction (PCR) analysis. The primers are designed to amplify a left arm and a right arm specifically for the cells that carry correct knock-in. PCR amplification for each single cell clone is conducted and PCR products are analyzed by gel electrophoresis to identify knock-in candidate clones with desired copy number.
c) Sequencing each candidate clone to verify for correct insertion of the desired fragment.

Step 4: Expanding verified clones into a colony

This step comprises:
a) Expanding verified single cell clones with proper knock-in to at least 5 million cell lines and cryopreserving the expanded single cell clones. Typically, the cell lines are expanded to 50 million. Furthermore, the extracted genomic DNA is used for further experiments.

In the CRISPR/Cas 9 approach, a genetic variant is inserted to a single or multiple safe harbor site(s) on a chromosome. The guide ribonucleic acid (gRNA) that specifically targets the AAVS1 locus is designed and cloned into a Cas9/gRNA expression vector. A donor vector is constructed. The donor vector comprises homologous arms to the AAVS1 locus and a knock-in cassette that comprises target sequence and antibiotic selection marker. The donor vector and gRNA plasmid is co-transfected into the target cells. After antibiotic selection, single cells that carry target sequence are identified. The safe harbor sites comprise AAVS1, human gene trap (ROSA)26S or locus (hROSA26), C—C chemokine receptor type 5 (CCR5), and Citrate Lyase Beta-Like (CLYBL). The size of the DNA insert is between about 20 to 200,000 base pairs in length. Multiple DNA fragments containing either the same or different SNV, INDEL, fusions or structural variant sequences are joined together as part of the insert.

In some cases, the same DNA sequences are tandemly joined either together, or at repeated intervals as part of the insert. Single, or multiple same, or different DNA fragments containing unique non-human sequences exist as part of the insert. The number of copies of the unique non-human sequence is at a fixed ratio either to the number of copies of the SNV, INDEL or fusions or structural variants, or to the number of copies of one or more of different non-human sequences. The allele frequency of the SNV, INDEL, fusions or structural variants is determined by PCR-based (quantitative PCR, or digital PCR) method and the fixed ratio exists in the insert. The method described herein creates cell lines of multiple genetic variants for use as models or reference standards for cancer or genetic disorders.

In some cases, the DNA sequences are inserted as part of an expression cassette which comprises promoter sequences and other sequences necessary for expressing mRNAs which contain variant sequences and unique non-human sequences. One or multiple copies of these expression cassettes are inserted.

Also disclosed herein is a method that allows specifically engineered quantitative marker sequences to accurately reflect the copy numbers of the inserted SNV, INDEL and fusions or structural variant sequences harbored in a single cell line. This allows accurate measurement of the ratio or allele frequencies of the genetic variants in the cell. The method comprises the following steps:

Step 1: Selecting many short stretches of specific DNA sequences such that none of the short stretches have identical consecutive fifteen or more deoxyribonucleotides to any DNA sequences in the human genome. Typically, the number of consecutive nucleotides in the specific DNA sequences is between thirteen and twenty-five. However, it can be between 5 to 5000 nucleotides long. These are called “unique non-human sequences”.

Step 2: Joining one or more of the unique non-human sequences with each other or with other specific sequences, such as the inserted SNV, INDEL and fusions or structural variant sequences to form one or more amplicons. Based on the unique non-human sequences on the amplicon, the amplicons can be qualitatively or quantitatively recognized, probed or counted by amplification methods such as polymerase chain reaction (PCR), a Next Generation Sequencing (NGS), an isothermal amplification or nucleic acid hybridization methods based on DNA-based or ribonucleic acid (RNA) based probes.

Step 3: Joining two or more of the amplicons with DNA sequences of any length which contain one or more of the specific sequences, such as the inserted SNV, INDEL and fusions or structural variant sequences to form a large linear DNA fragment of any length. Within the large DNA fragment, the number of identical copies of any of the specific sequences is at a fixed numeric ratio with each amplicon sequence. Each specific sequence has the same or different ratio with more than one amplicon sequences. Vice versa, each amplicon sequence has the same or different ratio with more than one specific sequence.

Step 4: Transfecting one or more of the large linear DNA fragments are transfected into mammalian cells either directly or via a vector, such as a plasmid or a form of naked or encapsulated virus-like nucleic acids. The transfected large linear DNA fragments exist either in a cytoplasm or in a nucleus as either episomal nucleic acids or integrated DNA on the chromosome. The transfected large linear DNA fragments are used transiently or replicated and propagated in a host cell line. The transfected cells harboring the large linear DNA or mRNA fragments or their derivative nucleic acids serve as a mimic of a native cell harboring specific sequences, such as the inserted SNV, INDEL and fusions or structural variant sequences.

Step 5: Storing and preserving the transfected cells similar to the native cells via spiking into whole blood, plasma, storage buffer, formalin-fixed paraffin-embedded (FFPE) (FFPE), etc. The transfected large linear DNA or mRNA fragments or their derivative nucleic acids are processed either the same way as the host cell genomic DNA, or mRNA or extracted together with the genomic DNA or mRNA of the host cell to serve as either cell-based or nucleic acid-based sample for further amplification- or hybridization-based molecular analysis.

Step 6: Processing the transfected large linear DNA fragments or their derivative nucleic acids into cell-free DNA fragments of a particular size, such as in the range from 50 to 600 base pairs or mRNAs. The processed cell-free DNA or mRNA fragments are either naked or complexed with DNA or mRNA-binding proteins, nucleosomes or exosomal vesicles. They are stored or preserved similar to the native cell-free DNA or mRNA via spiking into whole blood, plasma, urine, saliva or buffer. They are extracted together with the cell-free genomic DNA or mRNA to serve as sample for further amplification or hybridization based molecular analysis. For mRNA analysis, extraction methods for total RNA or mRNA are used in order to prepare for RNA analysis.

The amplification or hybridization based molecular analysis comprises, but is not limited to, the polymerase chain reaction (PCR), Next Generation Sequencing (NGS), quantitative PCR (qPCR), digital PCR (dPCR), droplet digital PCR (ddPCR), isothermal amplification methods such as rolling cycle amplification, nucleic acid hybridization methods such as DNA/RNA array, in situ hybridization, etc. For mRNA analysis, reverse transcriptase based methods are used.

In a non-limiting example, a non-human sequence “X” is ACTGACTGACTGACTGACTGACTG (SEQUENCE ID No. 1), a second non-human sequence “Y” is AAAACCCCAAAACCCCTTTTGGGG (SEQUENCE ID No. 2), and a third non-human sequence “Z” is TCGATCGATCAGTATCGATCGA (SEQUENCE ID No. 3).

A variant sequence A is:

(SEQUENCE ID No. 4)
AAGCCTACGTGATGGCCAGCGTGGACAACCCCCACGTGTGCCGCCT
GCTGGGCATCTGCCTCACCTCCACCGTGCAGCTCATCATGCAGCTC
ATGCCCTTCGGCTGCCTCCTGGACTATGTCCGGGAACACAAAGACA
ATATTGGCTCCCAGTACCTGCTCAACT
GGTGT.

A variant sequence B is:

(SEQUENCE ID No. 5)
GACCGTCGCTTGGTGCACCGCGACCTGGCAGCCAGGAACGTACTGG
TGAAAACACCGCAGCATGTCAAGATCACAGATTTTGGGCGGGCCAA
ACTGCTGGGTGCGGAAGAGAAAGAATACCATGCAGAAGGAGGCAAA
GTAAGGAGGTGGCTTTAGGTCAGCCAGCATTT.

A variant sequence C is:

(SEQUENCE ID No. 6)
CGGTTCTAATATAGTCACATTTTCATTATTTTTATTATAAGGCCTG
CTGAAAATGACTGAATATAAACTTGTGGTAGTTGGAGCTGATGGCG
TAGGCAAGAGTGCCTTGACGATACAGCTAATTCAGAATCATTTTGT
GGACGAATATGATCCAACAATAGAGGTAAATCTT.

A constructed insert sequence may comprise multiple number of single or repeated X, Y, Z, A, B or C, in a predesigned ratio between XYZ and ABCs. For one example, the construct can be XAYBZCXAYYZZZZ:

(SEQUENCE ID No. 7)
ACTGACTGACTGACTGACTGACTGAAGCCTACGTGATGGCCAGCGT
GGACAACCCCCACGTGTGCCGCCTGCTGGGCATCTGCCTCACCTCC
ACCGTGCAGCTCATCATGCAGCTCATGCCCTTCGGCTGCCTCCTGG
ACTATGTCCGGGAACACAAAGACAATATTGGCTCCCAGTACCTGCT
CAACTGGTGTAAAACCCCAAAACCCCTTTTGGGGGACCGTCGCTTG
GTGCACCGCGACCTGGCAGCCAGGAACGTACTGGTGAAAACACCGC
AGCATGTCAAGATCACAGATTTTGGGCGGGCCAAACTGCTGGGTGC
GGAAGAGAAAGAATACCATGCAGAAGGAGGCAAAGTAAGGAGGTGG
CTTTAGGTCAGCCAGCATTTTCGATCGATCAGTATCGATCGCGGTT
CTAATATAGTCACATTTTCATTATTTTTATTATAAGGCCTGCTGAA
AATGACTGAATATAAACTTGTGGTAGTTGGAGCTGATGGCGTAGGC
AAGAGTGCCTTGACGATACAGCTAATTCAGAATCATTTTGTGGACG
AATATGATCCAACAATAGAGGTAAATCTTACTGACTGACTGACTGA
CTGACTGAAGCCTACGTGATGGCCAGCGTGGACAACCCCCACGTGT
GCCGCCTGCTGGGCATCTGCCTCACCTCCACCGTGCAGCTCATCAT
GCAGCTCATGCCCTTCGGCTGCCTCCTGGACTATGTCCGGGAACAC
AAAGACAATATTGGCTCCCAGTACCTGCTCAACTGGTGTAAAACCC
CAAAACCCCTTTTGGGGAAAACCCCAAAACCCCTTTTGGGGTCGAT
CGATCAGTATCGATCGTCGATCGATCAGTATCGATCGTCGATCGAT
CAGTATCGATCG.

The ratio of relative number of copies of the variants or non-human sequences in the constructed inserted sequence and the wild-type sequence (W), X:Y:Z:A:B:C:W, in the insert are 2:2:4:2:1:1:1. By simultaneously measuring the relative number of copies for each of them by qPCR, ddPCR, NGS, or hybridization-based assays, the allele frequency or ratio of each variant can be more accurately determined using weighted averaged copies than when a simple copy of one variant is inserted to the genome. For example, the allele frequency (AF) of variant B can be more certainly determined by:

% AF_{B}=%(#AF_{X}/2+#AF_{Y}/2+#AF_{Z}/4+#AF_{A}/2+#AF_{B}+#AF_{C})/(6*#AF_{W})

where “#” is measured relative copy of a variant or the wild-type sequences.

Alternatively, non-human sequences and wild-type sequence are X, Y, Z and W, as above. A variant sequence is a DNA fragment that contains an expression cassette (EC) with promoters and other necessary sequences needed for mRNA expression in a cell. An example of a combination of different components is XYZ-EC-XYZ-EC where the ratio of sequence XYZ to EC is 1:1. A multiplexed copy number measurement by rt-qPCR, an NGS or a hybridization assay targeting any part of both XYZ and EC will improve the certainty of the result. For example, the allele frequency (% AF) of the expression cassette can be determined, with more confidence, by:

% AF_{EC}=%(#AF_{X}+#AF_{Y}+#AF_{Z}+#AF_{EC})/(4*#AF_{W})

where “#” is measured relative copy of a variant or the wild-type sequences.

The methods disclosed above are in no way to be construed as limiting of the method for creating reference cell lines with simultaneous genetic variants and accurate quantification of Allele frequency. While specific methods have been described herein, it is understood that the words, which have been used herein, are words of description and illustration, rather than words of limitation. Furthermore, the disclosed methods extend to all functionally equivalent methods, such as are within the scope of the appended claims. While specific embodiments are disclosed herein, it will be understood that those skilled in the art, having the benefit of the teachings of this specification, are capable of modifications and may affect other embodiments and changes thereto, without departing from the scope of the methods disclosed herein.

1. A method for simultaneously creating multiple Single Nucleotide Variant (SNV), insertions or deletions (INDEL), fusions or structural variant sequences harbored in a single cell line, comprising:
generating large sequence knock-in cell lines in a Adeno-Associated Virus Integration Site 1 (AAVS1) locus, or other safe harbor sites using a Clustered Regularly Interspaced Short Palindromic Repeats (CRISPR)/CRISPR-associated protein 9 (Cas9) gene editing system, comprising:
constructing a donor plasmid and guide Ribonucleic acid (gRNA), comprising:
determining a safe harbor gene to insert in a gene fragment sequence;
designing and cloning the gRNA targeting the safe harbor gene;
synthesizing a large piece of gene fragment sequence containing target sequences;
constructing a vector containing both sides of homologous recombination sequences at a CRISPR/Cas9 cutting site in the safe harbor gene, puromycin resistance gene, and promoters; and
cloning a synthesized gene fragment sequence into the constructed vector;
performing CRISPR/Cas 9 gene knock-in of cytosine-adenine-guanine (CAG) gene fragment sequence to a specific cell line, comprising:
selecting the gRNA with best cutting efficiency for the knock-in experiment, wherein both gRNA plasmid and donor vector are transfected into cells by one of chemical transfection and electroporation;
selecting the transfected cells for puromycin resistant cells that receive the donor plasmid in a target region; and
expanding the transfected cells after selection for checking presence of correct gene insertion;
generating and screening a single cell clone for a period ranging from 1-2 months, comprising:
sorting the cloned gene fragment sequence into 96-well plates, wherein a series of dilutions are performed to achieve single clone/per well, wherein three 96-well plates are used to screen for 100 single cell clones, wherein the wells are observed daily for first 3 days to record seeding of a single cell, and wherein ten to fifteen days are needed for cell expansion;
extracting genomic DNA from each cell clone for polymerase chain reaction (PCR) analysis, wherein primers are designed to amplify a left arm and a right arm for cells that carry a correct knock-in, wherein PCR amplification for each single cell clone is conducted and PCR products are analyzed by gel electrophoresis to identify knock-in candidate clones with desired copy number; and
sequencing each candidate clone to verify for correct insertion of a desired fragment;
expanding the verified single cell clones with proper knock-in into a colony of five to fifty million cell lines and cryopreserving the expanded clones; and
inserting a genetic variant to a single or multiple safe harbor site(s) on a chromosome, wherein size of the genomic DNA insert is between 20 to 200,000 base pairs in length, and wherein multiple DNA fragments containing either the same or different SNV, INDEL, fusions or structural variant sequences are joined together as part of the genomic DNA insert.

2. The method according to claim 1, wherein same DNA fragments are tandemly joined either together or at repeated intervals as part of the genomic DNA insert.

3. The method according to claim 1, wherein single or multiple, same or different DNA fragments containing unique non-human sequences exist as part of the insert.

4. The method according to claim 1, wherein the DNA fragments are inserted as part of an expression cassette comprising promoter sequences and other sequences necessary for expressing mRNAs which contain variant sequences and unique non-human sequences.

5. The method according to claim 1, wherein the method creates cell lines of multiple genetic variants for use as models or reference standards for cancer or genetic disorder.

6. The method according to claim 1, wherein allele frequency of the SNV, INDEL, fusions or structural variants is determined by nucleic-acid-amplification-based or isothermal-amplification-based methods.

7. The method according to claim 6, wherein the nucleic-acid-amplification-based method is any one selected from a conventional PCR, a real-time PCR, a Next Generation Sequencing (NGS), and a droplet digital PCR (ddPCR).

8. The method according to claim 6, wherein the isothermal-amplification-based method is a rolling-cycle-based method.

9. A method for engineering quantitative marker sequences to accurately reflect copy numbers of inserted Single Nucleotide Variant (SNV), insertions or deletions (INDEL), fusions or structural variant sequences harbored in a single cell line, comprising:
selecting one or more unique non-human sequences, wherein the selected unique non-human sequences do not have identical consecutive fifteen or more deoxyribonucleotides (DNA) sequences compared to similar DNA sequences in a human genome;
joining one or more of the unique non-human sequences to each other or with specific sequences comprising the inserted SNV, INDEL and fusions or structural variant sequences to form one or more amplicons, wherein the amplicons are qualitatively or quantitatively recognized, probed or counted by amplification methods comprising one of a polymerase chain reaction (PCR), a Next Generation Sequencing (NGS), an isothermal amplification, and a nucleic acid hybridization method based on DNA-based or ribonucleic acid (RNA) based probes;
joining two or more of the amplicons with the DNA sequences comprising one or more of the specific sequences comprising the inserted SNV, INDEL and fusions or structural variant sequences to form a large linear DNA fragment, wherein number of identical copies of any of the specific sequences is at a fixed numeric ratio with each amplicon sequence within the large linear DNA fragment, wherein each specific sequence can have same or different ratio with more than one amplicon, and wherein each amplicon can have same or different ratio with more than one of the specific sequences;
transfecting one or more of the large linear DNA fragments into mammalian cells either directly or via a vector comprising one of a plasmid and a form of naked or encapsulated virus-like nucleic acid, wherein the transfected large linear DNA fragments exist in one of a cytoplasm or in a nucleus as one of episomal nucleic acid or an integrated DNA on a chromosome, wherein the transfected large linear DNA fragments are one of used transiently and replicated and propagated in a host cell line, and wherein the transfected cells harboring the large linear DNA or mRNA fragments or their derivative nucleic acids serves as a mimic of a native cell harboring the specific sequences comprising the inserted SNV, INDEL and fusions or structural variant sequences;
storing or preserving the transfected cells similar to the native cells via spiking into one of whole blood, plasma, storage buffer, and Formalin-fixed Paraffin-embedded (FFPE), wherein the transfected large linear DNA or mRNA fragments or their derivative nucleic acids are either processed by either same way as a host cell genomic DNA or mRNA or extracted together with the host cell genomic DNA or mRNA to serve as either cell-based or nucleic acid-based sample for further amplification or hybridization based molecular analysis; and
processing the transfected large linear DNA fragments or their derivative nucleic acids into cell-free DNA fragments of a size in the range from 50 to 600 base pairs or mRNAs, wherein the processed cell-free DNA or mRNA fragments are either naked or complexed with DNA/mRNA-binding proteins, nucleosomes or exosomal vesicles, wherein the processed cell-free DNA or mRNA fragments are stored or preserved similar to the native cell-free DNA or mRNA via spiking into one of whole blood, plasma, urine, saliva, and buffer, and wherein the processed cell-free DNA or mRNA fragments are extracted together with the cell-free genomic DNA or mRNA to serve as sample for further amplification or hybridization based molecular analysis.

10. The method according to claim 9, wherein the amplification or hybridization based molecular analysis is performed either by Polymerase Chain Reaction (PCR), Next Generation Sequencing (NGS), quantitative PCR (qPCR), digital PCR (dPCR), droplet digital PCR (ddPCR), or isothermal amplification, rolling cycle amplification, or nucleic acid hybridization, DNA/RNA array, or in-situ hybridization.


SEQUENCE LISTING (7 sequences)

SEQ ID NO: 1 - DNA, 24 bp, Artificial Sequence
  Description of Artificial sequence Non-human sequence
actgactgactgactgactgactg

SEQ ID NO: 2 - DNA, 24 bp, Artificial Sequence
  Description of Artificial sequence Non-human sequence
aaaaccccaaaaccccttttgggg

SEQ ID NO: 3 - DNA, 22 bp, Artificial Sequence
  Description of Artificial sequence Non-human sequence
tcgatcgatcagtatcgatcga

SEQ ID NO: 4 - DNA, 170 bp, Artificial Sequence
  Description of Artificial sequence Variant sequence A
aagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgt

SEQ ID NO: 5 - DNA, 170 bp, Artificial Sequence
  Description of Artificial sequence Variant sequence B
gaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtaaggaggtggctttaggtcagccagcattt

SEQ ID NO: 6 - DNA, 172 bp, Artificial Sequence
  Description of Artificial sequence Variant sequence C
cggttctaatatagtcacattttcattatttttattataaggcctgctgaaaatgactgaatataaacttgtggtagttggagctgatggcgtaggcaagagtgccttgacgatacagctaattcagaatcattttgtggacgaatatgatccaacaatagaggtaaatctt

SEQ ID NO: 7 - DNA, 886 bp, Artificial Sequence
  Description of Artificial sequence Constructed Insert Sequence
actgactgactgactgactgactgaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtaaaaccccaaaaccccttttgggggaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtaaggaggtggctttaggtcagccagcattttcgatcgatcagtatcgatcgcggttctaatatagtcacattttcattatttttattataaggcctgctgaaaatgactgaatataaacttgtggtagttggagctgatggcgtaggcaagagtgccttgacgatacagctaattcagaatcattttgtggacgaatatgatccaacaatagaggtaaatcttactgactgactgactgactgactgaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtaaaaccccaaaaccccttttggggaaaaccccaaaaccccttttggggtcgatcgatcagtatcgatcgtcgatcgatcagtatcgatcgtcgatcgatcagtatcgatcg